(19) United States
(12) Patent Application Publication
(10) Pub. No.: US 2005/0142219 A1
(43) Pub. Date: Jun. 30, 2005

(54) Essentially sodium-free potassium salt derived from seaweed

(75) Inventors: Dilum D. Dunuwila, Okemos, MI (US); Kris A. Berglund, Okemos, MI (US)

Correspondence Address: McLEOD & MOYNE, P.C., 2190 Commons Parkway, Okemos, MI, 48864, US

(73) Assignee: Diversified Natural Products, Inc., East Lansing, MI (US)

(21) Appl. No.: 11/017,878

(22) Filed: Dec. 21, 2004

Related U.S. Application Data
(60) Provisional application No. 60/532,478, filed on Dec. 26, 2003.

(51) Int. Cl. A61K 35/80; A61K 33/00

(52) U.S. Cl. 424/722; 424/195.17

(57) ABSTRACT

A potassium salt composition from seaweed, methods for producing the composition, and methods of using the composition of an essentially sodium-free salt substitute and/or potassium dietary supplement is described. The potassium salt composition has a pleasant salty taste and has a crystalline appearance that resembles regular table salt, but it is essentially free of the sodium and the metals. In addition, the product is essentially free of seaweed aromas or flavors which are removed by absorption or adsorption from an aqueous solution containing the dissolved potassium salt substitute.

### CROSS-REFERENCE TO RELATED APPLICATIONS

This application claims priority to provisional application Ser. No. 60/532,478 filed Dec. 26, 2003.

### STATEMENT REGARDING FEDERALLY SPONSORED RESEARCH OR DEVELOPMENT

Not Applicable

### BACKGROUND OF THE INVENTION

(1) Field of the Invention

The present invention relates generally to purified sea salt dietary supplements, and more particularly to essentially sodium-free potassium sea salt compositions. Specifically, the present invention relates to potassium salt compositions and methods of making them, which have been isolated from the surface of dried seaweeds.

(2) Description of the Related Art

Many individuals suffer from hypertension that is related to excessive salt consumption. There is a need to reduce salt intake by replacement of regular salt with sodium-free salty flavors. In addition, many individuals have a potassium deficiency in their diets. Various low-sodium and sodium-free salt substitutes have been developed to give foods salty flavors with reduced sodium. Often potassium salts are included which allow for the reduction of sodium, and improve the potassium levels in the diet.

U.S. Pat. No. 5,527,959 to Berglund et al. teaches a process of admixing lysine hydrochloride and a succinate compound, heating the solution at a reduced pressure, cooling to form a crystalline composition, and separating the composition from solution to prepare the composition. U.S. Pat. No. 5,565,610 to Berglund et al. teaches the composition LysₐCl_{b}Succ_{c}X_{d} prepared by this process.

U.S. Pat. No. 5,897,908 to Berglund et al. teaches a physical mixture having a salty taste which consists essentially of lysine monohydrochloride and potassium chloride alone or admixed with small amounts of succinic acid, in particular weight ratios. The mixture masks the bitter aftertaste of the potassium chloride and provides dietary lysine.

U.S. Pat. No. 5,626,904, to Frederiksen et al. teaches a salt product comprising particles of NaCl, K/Mg-double salts and optionally KCl, homogeneously distributed throughout each particle. The product may be obtained from an aqueous salt mixture, preferably a solution containing the desired proportion of salt ions and having a water content of 50 to 90% by weight by subjecting the aqueous salt mixture to instant drying at a temperature of 70° to 200° C. for a period of 1 to 60 seconds to obtain a dry product.

U.S. Pat. No. 5,871,803 to Bonorden et al. teaches a salt flavor enhancing composition consists essentially of a combination of sodium chloride and potassium chloride and a combination of magnesium chloride and magnesium sulfate. The composition can include a combination of sodium ions, chloride ions, potassium ions, magnesium ions, and sulfate ions.

U.S. Pat. No. 6,541,050 to Bonordon et al. teaches a salt flavor enhancing composition which includes a combination of sodium chloride and potassium chloride in a range of about 74 to about 99 percent by weight and sulfate-containing salts or a combination of sulfate-containing salts and chloride-containing salts in a range of about 1 to about 26 percent by weight. The sulfate-containing salts can be potassium sulfate, calcium sulfate, or combinations thereof. The chloride-containing salts may be calcium chloride, magnesium chloride, or combinations thereof. The composition also can be adapted for use on food products having a high intrinsic sodium content. The composition can include potassium chloride in a range of about 75 to about 95 percent by weight, and a sulfate-containing salt or a combination of a sulfate-containing salt and a chloride-containing salt in a range of about 5 to 25 percent by weight. The sulfate-containing salts can be potassium sulfate, calcium sulfate, and combinations thereof.

Low-sodium salts have also been isolated directly from sea water. U.S. Pat. No. 6,048,569 to Garcia et al. teaches a comestible liquid sea salt preparation and method of production. The comestible preparation is produced from sea water and has a sodium content of about 9.8 wt %. The method for producing the preparation involves a series of steps in which sea water is decanted, evaporated, converted into a spray, and concentrated. The preparation also undergoes a micro plankton regulation step, and sodium chloride is added to the resulting preparation so that the concentration of sodium chloride is 25% of the total preparation.

Other approaches for extracting sea water salts use seaweed to extract the desired salts. In Japan, traditional methods of extracting salt from sea water include using seaweed ashes. A water solution was also used in the earthenware vessels to produce the salt.

Thus, the related art describe various formulations of low-sodium salts. Garcia et al. further teach sea water as an inexpensive source of the salts. Additionally, Japanese traditions include salt-making processes which derive sea water salts using seaweed. While the related art improve upon basic table salt compositions and suggest inexpensive sources for these salts, there still exists a need for high quality food grade potassium salts and a method of providing the compositions from inexpensive sources to meet the combined need for essentially sodium-free salts and increased potassium in human diets.

### OBJECTS

Therefore, it is an object of the present invention to provide a composition isolated from seaweed which can be used as an essentially sodium-free salt substitute/potassium dietary supplement.

It is further an object of the present invention to provide a method for isolating the potassium salt composition from seaweed.

These and other objects will become increasingly apparent by reference to the following description.

### SUMMARY OF THE INVENTION

The present invention provides a comestible composition which comprises a crystalline potassium salt composition which has been isolated from the surface of dried ocean harvested seaweed and is essentially free of sodium, magnesium, calcium, and heavy metals from the seaweed or salt water from which the seaweed is removed. In preferred embodiments, the composition consists essentially of the salt in a dried form. In preferred embodiments the composition comprises less than 10 ppm of lead. In some embodiments the composition comprises from about 50% to about 53% potassium by weight.

The present invention further provides a method for providing a salty taste to a food which comprises incorporating into the food an effective amount of a comestible composition which comprises a crystalline potassium salt composition which has been isolated from the surface of dried ocean harvested seaweed and is essentially free of sodium, magnesium, calcium, and heavy metals from the seaweed or salt water from which the seaweed is removed.

The present invention further provides a method for isolating a comestible dried crystalline potassium salt composition which comprises removing an impure salt composition from a surface of dried seaweed, dissolving the impure salt in an aqueous solution, separating particulate impurities from the aqueous solution, treating the resulting aqueous solution so as to remove flavor and color from the solution, crystallizing the salt composition from the solution, removing the composition from the solution, and drying the salt composition to produce the dried potassium salt composition. In preferred embodiments of the method, the treating is with an agent which removes flavor, color, and aroma compounds.

In further embodiments the agent is activated carbon. In still further embodiments the impure salt is dissolved in an aqueous solution at a concentration of 10% to 40% by weight. In still further embodiments the particles are separated from the aqueous solution by filtration, hydrocyclone, or centrifugation. In still further embodiments the salt composition is removed from the solution by filtration. Preferably, the method further comprises preparing a saturated aqueous solution of the dried potassium salt composition and recrystallizing the saturated aqueous solution of the potassium salt composition to provide a crystalline potassium salt composition. In preferred embodiments of the method the crystalline potassium salt composition comprises crystals from about 200 microns to about 1000 microns in size.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control. Definitions for the following terms are provided to promote a further understanding of the present invention.

The phrase “essentially free of sodium” refers to a composition which contains less than 1000 ppm sodium.

The phrase “essentially free of” is defined as less than 1000 ppm of any metal.

The term “seaweed” as used herein refers to any seaweed, such as red algae, brown algae, green algae, and sea grasses, including Lithothamnion (red), Laminaria (brown), Porphyra (red), Undaria (brown), Kappaphycus (red), Gracilaria (red), Ascophyllum (brown), Eucheuma (red), Macrocystis (brown), Lessonia (brown), Hizikia (brown), Hypnea, Enteromorpha (green), and Cladosiphon (brown). Other seaweeds encompassed by the term include Gelidium (red), Chondrus (red), Sargassum (brown), Gigartina (red), Iridaea (red), Alaria, Ahnfeltia (red), Durvillaea (brown), Cladosiphon (brown), Ulva (green), Meristotheca, Monostroma (green), Caulerpa (green), Mastocarpus (red), Pterocladia (red), Ecklonia (brown), Turbinaria (brown), Gelidiella (red), Gloiopeltis (red), Palmaria (red), Codium (green), Furcellaria (red), Fucus (brown), Nereocystis (brown), Eisenia, and Himanthalia. Porphyra algae (nori) which are commonly cultivated include the Porphyra species abbotae, acanthophora, atropurpurae, columbina, crispata, fallax, haitanensis, kuniedae, leucostica, perforata, pseudolanceolata, seriata, spirilis, suborticulata, tenera, torta, umbilicalis, vietnamensis, and yezoensis. Undaria (brown algae) known as “wakame” include such species as pinnitifida and peterseniana. Laminaria (brown algae) is kelp and is known as “kombu” including species such as angustata, bongardiana, diabolical, digitata, groenlandica, hyperborea, japonica, longicruris, longissima, ochroleuca, octotensis, religiosa, saccharina, setchelli, and sachinzii.

Potassium is found on land in deposits in the form of potassium chloride, commonly referred as potash, in the mineral sylvite. Ores such as sylvanite contain a mixture of slyvite and sodium chloride. However, potassium found on land usually contains clays and impurities, and is found as a mixture of white and red crystals. The isolated potash is used for fertilizer or chemical production. Potash can be underground mined or solution mined, where a brine is used for recrystallization. The ore is crushed, ground, and agitated in a slurry to remove clay. The slurry is screened and cycloned to remove the brine and clay. Next, the potash is separated from other salts by flotation before it is centrifuged and graded. Some potash is recrystallized to refine the potassium chloride.

The oceans of the world contain an immense reservoir of salts, and are increasingly harvested as a source of seaweed derived biopolymers. Biopolymers including alginic acid, agar, and carrageenan are utilized in foods and drugs. Algae and sea grasses are classified by morphology, chemistry and ultrastructure. The seaweeds are grouped as red algae, brown algae, green algae, and sea grasses. Porphyra are one type of algae which are commonly cultivated. These are used as food in the form of Nori, which is used for flavoring or in sushi, in growing demand across the world. The harvested algae are washed in freshwater to remove sediments, chopped, and dried to prepare the Nori.

In the first stage of processing for the production of biopolymers and other compounds the seaweed is dried. During this drying process a significant amount of material is expressed out of the seaweed and crystallizes upon the surface of it. Usually, this crystalline material is physically separated from the seaweed and treated as waste. The current process uses this discarded material as a feedstock and transforms it into a valuable essentially sodium-free salt substitute/potassium dietary supplement for human consumption.

In the embodiments of the present invention in the following Examples, the process first began with an initial recovery step. Seaweed was air dried and the resulting solid material that formed upon the surface was removed by shaking. Secondly, the material underwent a pretreatment step for the purpose of removing solids. In this step, the solid materials recovered from the seaweed drying were dissolved into water at about 10-40% by weight. The resulting solution was then filtered to remove insoluble materials such as sand and dirt. In other embodiments, solid-liquid separation devices such as a hydrocyclone or a centrifuge can be used to remove insoluble materials.

Thirdly, the resulting solution was treated with food grade activated carbon to remove undesired color, flavor, and aroma compounds from the seaweed. In other embodiments absorbants or adsorbants such as resins, zeolites and clays can be used. Further, other separation methods such as floatation can be used. Fourthly, once these compounds were removed then the product was crystallized. In this step, the product was recovered from the solution by a controlled evaporation of the water. The size distribution of the resulting crystals can be controlled by the rate of evaporation. The final step was finishing, wherein the resulting slurry was filtered to remove the product crystals and the product crystals were dried. The product was a white, crystalline material which had the appearance of regular table salt. The resulting product was essentially free of sodium, essentially free of heavy metals, and comprised approximately 50-53% potassium by weight. It had a desirable salty taste.

Potassium chloride has a wide range of uses in foods, nutritional products, and pharmaceuticals. Pharmaceutical uses include using the salt for oral electrolyte maintenance to prevent dehydration, or as a potassium source for hemodialysis or intravenous uses. Intravenous uses of potassium chloride are necessary in patients with severe hypokalemia. Hypokalemia can result from diabetic ketoacidosis, severe diarrhea, and rare adrenal disorders. Hypokalemia can also be the result of the use of strong diuretics. The salt is also used to treat digitalis intoxication associated with overdoses of digoxin and digitoxin used to treat congestive heart failure, tachycardia, and atrial fibrillation. Deficiencies of potassium can also lead to weakness, fatigue, paralysis, and kidney dysfunction.

### EXAMPLE 1

### Use of Activated Carbon Column Absorption

Twenty grams of the crude material from seaweed was slurried with 200 ml of water to dissolve soluble components. The slurry was allowed to settle and the sediment was separated from the solution by decantation. The decanted solution was vacuum filtered using a paper filter to remove larger particles and using a glass fiber filter to remove “micro” particles. The solution was passed through an activated carbon column, containing 3.0 grams of activated carbon, to remove undesirable color and odor. The salt substitute/potassium dietary supplement was crystallized by evaporative crystallization. The dry weight of the product was 15.4 grams.

### EXAMPLE 2

### Use of Activated Carbon and Filtering

Twenty grams of the crude material from seaweed was slurried with 200 ml of water to dissolve soluble components. The slurry was allowed to settle and the sediment was separated from the solution by decantation. The decanted solution was vacuum filtered using a paper filter to remove larger particles and using a glass fiber filter to remove “micro” particles. The solution was slurried in 3.0 grams of activated carbon to remove undesirable color and odor. The slurry was vacuum filtered to remove activated carbon using a paper filter. The salt substitute/potassium dietary supplement was crystallized by evaporative crystallization. The dry weight of the product was 15.3 grams.

### EXAMPLE 3

### Use of Seeding for Crystallization

A saturated aqueous solution of seaweed salt was prepared using about 2.0 grams of product from Example 4. The solution was seeded using a small amount (0.1 grams) of product from Example 4. The seeded solution was evaporated in open air to dryness. Crystals ranging from 200-1000 microns in size were obtained.

### EXAMPLE 4

### Analysis

The final product of the above process was analyzed for heavy metals reported as lead. There was no detection at the 10 ppm level. Similarly, there was no detection of sodium at the 10 ppm level.

### EXAMPLE 5

### Taste Test

The product of the above process was tasted by four individuals. All agreed that it has an agreeable, salty taste. No seaweed aromas or flavors were detected.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A comestible composition which comprises a crystalline potassium salt composition which has been isolated from the surface of dried ocean harvested seaweed and is essentially free of sodium, magnesium, calcium, and heavy metals from the seaweed or salt water from which the seaweed is removed.

2. The composition of claim 1 which consists essentially of the salt in a dried form.

3. The composition of claim 1 wherein the composition comprises less than 10 ppm of lead.

4. The composition of claim 1 wherein the composition comprises from about 50% to about 53% potassium by weight.

5. A method for providing a salty taste to a food which comprises incorporating into the food an effective amount of a composition which comprises a crystalline potassium salt composition which has been isolated from the surface of dried ocean harvested seaweed and is essentially free of sodium, magnesium, calcium, and heavy metals from the seaweed or salt water from which the seaweed is removed.

6. A method for isolating a comestible dried crystalline potassium salt composition which comprises:
(a) removing an impure salt composition from a surface of dried seaweed;
(b) dissolving the impure salt in an aqueous solution;
(c) separating particulate impurities from the aqueous solution;
(d) treating the aqueous solution of step (c) so as to remove flavor and color from the solution;
(e) crystallizing the salt composition from the solution;
(f) removing the composition from the solution; and
(g) drying the salt composition to produce the dried potassium salt composition.

7. The method of claim 6 wherein the treating is done with an agent which removes flavor, color, and aroma compounds.

8. The method of claim 7 wherein the agent is activated carbon.

9. The method of claim 6 wherein the impure salt is dissolved in an aqueous solution at a concentration of 10% to 40% by weight.

10. The method of claim 6 wherein the particles are separated from the aqueous solution by filtration, hydrocyclone, or centrifugation.

11. The method of claim 6 wherein the salt composition is removed from the solution by filtration.

12. The method of claim 6 further comprising preparing a saturated aqueous solution of the dried potassium salt composition and recrystallizing the saturated aqueous solution of the potassium salt composition to provide a crystalline potassium salt composition.

13. The method of claim 12 wherein the crystalline potassium salt composition comprises crystals from about 200 microns to about 1000 microns in size.
